(19) United States
(12) Patent Application Publication
(10) Pub. No.: US 2024/0216381 A1
(43) Pub. Date: Jul. 4, 2024

(54) Px-cholorogenic Acid Comps

(71) Applicant: PX ING, LLC, Lewisville, TX (US)

(72) Inventors: Ralf Jäger, Whitefish Bay, WI (US); Martin Purpura, Spring, TX (US); Shawn D. Wells, Frisco, TX (US); Kylin Liao, Plano, TX (US)

(21) Appl. No.: 18/393,293

(22) Filed: Dec. 21, 2023

Related U.S. Application Data
(60) Provisional application No. 63/434,206, filed on Dec. 21, 2022.

(51) Int. Cl. A61K 31/522; A61K 31/216; A61P 25/28

(52) U.S. Cl. CPC A61K 31/522; A61K 31/216; A61P 25/28

(57) ABSTRACT

The disclosed compositions, systems and methods relate to compositions for human consumption and comprise a combination of paraxanthine and/or 1-methylxanthine and chlorogenic acid and optionally other compounds that modulate the effects of a combination of paraxanthine and/1-methylxanthine and chlorogenic acid. Further disclosed are methods of use of the foregoing compositions for improvement of at least one of cognitive performance, mood, and/or sleep.

### CROSS-REFERENCE TO RELATED APPLICATION(S)

This application claims priority to U.S. Provisional Application No. 63/434,206 filed Dec. 21, 2022, and entitled “BIOACTIVE COMPOSITIONS AND METHODS OF USE THEREOF,” which is hereby incorporated by reference in its entirety under 35 U.S.C. § 119(e).

### TECHNICAL FIELD

The disclosed technology relates generally to compositions, methods, for physiologic and/or cognitive functions through administration of compositions containing combinations of paraxanthine and chlorogenic acid.

### BACKGROUND

Thus, there is a need in the art to identify alternative chemical compounds and mixtures thereof that may provide benefits. It is also desirable to provide chemical compounds and mixtures thereof that may be used to provide a variety of benefits, and in particular, synergistic benefits.

### BRIEF SUMMARY

Disclosed herein are compositions comprising paraxanthine and/or 1-methylxanthine and chlorogenic acid. In exemplary implementations, the composition comprises about 50 mg to about 400 mg of paraxanthine and/or 1-methylxanthine and at least about 25 mg chlorogenic acid. In further implementations, the composition comprises chlorogenic acid in an amount of from about 25 mg to about 500 mg. In further embodiments, the composition may further comprise one or more metabolites of paraxanthine.

Further disclosed here is a method of treating a condition in a subject in need thereof, may include administering to the subject the composition disclosed herein. A method of enhancing attention in a subject in need thereof may include administering the composition disclosed herein. A method of improving working memory in a subject in need thereof may include administering a composition to the subject may include the composition disclosed herein. A method of improving cognitive performance in a subject may include administering the composition disclosed herein. A method of preventing or treating TBI in a subject in need thereof. A method of treating an inflammatory disease or condition in a subject in need thereof. A method for improving joint health in a subject in need thereof. A method for enhancing neuroplasticity in a subject in need thereof. A method for improving digestive health in a subject in need thereof. A method for attenuating hypertension in a subject in need thereof. A method of modulating cellular energy homeostasis may include administering to a subject the composition disclosed herein. A method of modulating blood glucose levels in a subject by administering to a subject composition disclosed herein.

In certain embodiments, the disclosed compositions comprise a further active ingredient, selected from a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (Sadenosylmethionine), ASU (avocado-soybean unsapponifiable fraction), cetyl myristoleate, Dolichos falcate, triterpenoids, Acacia catechu, Andrographis paniculata, Scutalleria baicalensis, Agmatine sulfate, Stinging Nettle, Sea Buckthorn, Curcumin, Cissus Quadrilangularis, Boswellia serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavonoids, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-tyrosine, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, Ornithine, Citrulline, Pyruvate, Eleutherococcus senticosus, D-Ribose, whey protein, Trimethylglycine, Arginine, HMB (β-hydroxy β-methylbutyrate), milk protein, Schisandra chinensis, Leucine, Betalains, Leucic Acid, L-Carnitine, Sodium Bicarbonate, Arachidonic acid, Beta-Alanine, Brassinosteroids, Hemp Protein, Alanylglutamine, Rhaponticum carthamoides, Casein, Ecdysteroids, Creatine, Branched-Chain Amino Acids, Beetroot, Coffee, Nitrate, Panax ginseng, Clenbuterol, Alpha-GPC, Valine, Colostrum, Trichopus zeylanicus, Ashwagandha, Terminalia arjuna, Eggs, Ursolic Acid, Isoleucine, medium-chain triglycerides, Glutamine, zinc, vitamin D, maca, Schizandra, nicotinamide mononucleotide (NMN), exogenous ketones, Ergothioneine, berberine, dihydroberberine and combinations thereof.

In certain implementations, ratio of the amount of paraxanthine and/or 1-methylxanthine and chlorogenic acid administered to the subject is from about 1:10 to about 1:30. In further implementations, ratio of the amount of paraxanthine and chlorogenic acid administered to the subject is from about 1:10 to about 1:10.

In certain embodiments, the composition is substantially free a caffeine.

Further disclosed herein is a method of improving cognitive function in a subject comprising administering to the subject a composition comprising an effective amount of paraxanthine and chlorogenic acid. In certain embodiments, improved cognitive function in the subject is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition. In certain embodiments, administration of the composition to the subject enhances mood in the subject. In further embodiments, administration of paraxanthine and chlorogenic acid produce a synergistic enhancement in cognitive function in the subject, relative to the administration of paraxanthine or chlorogenic acid alone.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE FIGURES

None.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “cardiovascular disease” or “cardiovascular disorder” is used herein in the broadest sense and includes all diseases and pathological conditions the pathogenesis of which involves abnormalities of the blood vessels, such as, for example, atherosclerotic plaque formation (including stable or unstable/vulnerable plaques), atherosclerosis, arteriosclerosis, arteriolosclerosis, and elevated systemic lipopolysaccharide (LPS) exposure. The term additionally includes diseases and pathological conditions that benefit from the inhibition of the formation of atherosclerotic plaques. Cardiovascular diseases include, without limitation, coronary artery atherosclerosis, coronary microvascular disease, stroke, carotid artery disease, peripheral arterial disease, ischemia, coronary artery disease (CAD), acute coronary syndrome (ACS), coronary heart disease (CHD), conditions associated with CAD and CHD, cerebrovascular disease, peripheral vascular disease, aneurysm, vasculitis, venous thrombosis, diabetes mellitus, and metabolic syndromechronic kidney disease, remote tissue injury after ischemia and reperfusion, cardiopulmonary bypass. Specifically included within this group are all cardiovascular diseases associated with the occurrence, development, or progression of which can be controlled by the inhibition of the atherosclerotic plaque formation.

As used herein, “improving serum lipid profile” means a reduction is serum total and/or LDL cholesterol and triacyl-glycerol levels, as well as increasing the ratio of HDL cholesterol to LDL cholesterol.

In certain aspects, disclosed herein are methods to improving serum lipid profile in a subject through the administration of an effective amount of one or more compositions disclosed herein.

The term “overweight” is defined as the condition wherein the individual has a BMI greater than or 25 kg/m2 and less than 30 kg/m2. The terms “overweight” and “pre-obese” are used interchangeably.

As used herein, the term “obesity” is defined as the condition wherein the individual has a BMI equal to or greater than 30 kg/m2. According to a WHO definition the term obesity may be categorized as follows: the term “class I obesity” is the condition wherein the BMI is equal to or greater than 30 kg/m2 but lower than 35 kg/m2; the term “class II obesity” is the condition wherein the BMI is equal to or greater than 35 kg/m2 but lower than 40 kg/m2; the term “class III obesity” is the condition wherein the BMI is equal to or greater than 40 kg/m2.

As used herein, the terms “manage,” “managing,” and “management” encompass preventing, delaying, or reducing the severity of a recurrence of an adipose associated body composition or body weight disorder, such as obesity, lypodystrophy, diabetes or metabolic syndrome, fibrosis and cancer in a patient who has already suffered from such a disease, disorder or condition. The terms encompass modulating the threshold, development, and/or duration of the adipose associated body composition or body weight disorder, such as obesity, lypodystrophy, diabetes or metabolic syndrome, fibrosis and cancer or changing how a patient responds to the adipose associated body composition or body weight disorder

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “synergistic effect” or grammatical variations thereof means and includes a cooperative action encountered in a combination of two or more active compounds in which the combined activity of the two or more active compounds exceeds the sum of the activity of each active compound alone.

The term “synergistically effective amount,” as used herein, means and includes an amount of two or more active compounds that provides a synergistic effect defined above.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, “cognitive function” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care, speed of processing, reasoning and problem solving and social cognition.

### Compositions

Disclosed are compositions comprising a combination of paraxanthine and chlorogenic acid and the related uses thereof. Paraxanthine may be produced synthetically or may be isolated from a natural source or through fermentation. Paraxanthine isolated from such sources may be purified to 95% or greater purity. Optionally, less purification may be used such that combination of paraxanthine for 50%, or even less, of the material. In some embodiments, it may be preferable to utilize paraxanthine isolated from a natural source which may include other congeners of paraxanthine typically found in paraxanthine sources.

Further disclosed are compositions comprising a combination of 1-methylxanthine (also referred to herein as “1-Mx”) and chlorogenic acid and the related uses thereof. 1-methylxanthine may be produced synthetically or may be isolated from a natural source or through fermentation. 1-methylxanthine isolated from such sources may be purified to 95% or greater purity. Optionally, less purification may be used such that combination of paraxanthine for 50%, or even less, of the material. In some embodiments, it may be preferable to utilize 1-methylxanthine isolated from a natural source which may include other congeners of 1-methylxanthine typically found in 1-methylxanthine sources.

In certain embodiments, the composition is formulated such that a dose contains paraxanthine and/or 1-methylxanthine ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

Chlorogenic Acid is a polyphenol and the ester of caffeic acid and quinic acid. Chlorogenic acid scavenges free radicals, which inhibits DNA damage and may protect against the induction of carcinogenesis. The chemical formula for CA is C₁₆H₁₈O₉. According to certain embodiments, Chlorogenic acid has the and the structure:

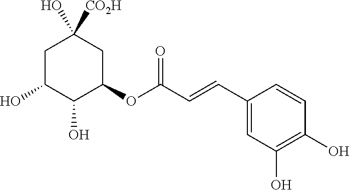 [SMILES: O=C(/C=C/c1ccc(O)c(O)c1)O[C@@H]1C[C@](O)(C(=O)O)C[C@@H](O)[C@H]1O]

In certain embodiments, the composition is formulated such that a dose contains chlorogenic acid ranging from about 25 to about 1000 mg (e.g., about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

In certain embodiments, the combination of paraxanthine and/or 1-methylxanthine and chlorogenic acid may be combined with one or more other chemical compounds (e.g. other active ingredients), to provide a plurality of positive effects in a subject. By altering the dosage of the combination of paraxanthine and chlorogenic acid and/or chemical compounds it is combined with, various physiological effects may be selected for. The compositions may provide primarily a single benefit, or may provide multiple benefits simultaneously. In certain embodiments, the combination of paraxanthine and chlorogenic acid is combined with one or more additional active ingredients selected from: a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, cocrystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (Sadenosylmethionine), ASU (avocado-soybean unsapponifiable fraction), cetyl myristoleate, Dolichos falcate, triterpenoids, Acacia catechu, Andrographis paniculata, Scutalleria baicalensis, Agmatine sulfate, Stinging Nettle, Sea Buckthorn, Curcumin, Cissus Quadrilangularis, Boswellia serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, tocopherols, theophylline, alphayohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, Ginkgo biloba, siberian ginseng, astragalus, licorice, green tea, reishi, dehydroepiandrosterone (DHEA), pregnenolone, N-acetyl-tyrosine, glucuronolactone, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, forskolin (Coleus forskohlli), 2-(dimethylamino)ethanol (DMAE), DMAE bitartrate, medium chain triglycerides, creatine, citrulline, arginine, lions mane, cordyceps, leucine, isoleucine, valine, BAIBA, ergothioncine, grains of paradise, Kanna, Huperzine A, ketones, Maca, ginseng, ashwagandha, rhodiola, theanine and combinations thereof.

In certain embodiments, paraxanthine and chlorogenic acid are present in about equal amounts. In these embodiments, paraxanthine and chlorogenic acid each comprise about 50% of the combined weight of paraxanthine and chlorogenic acid within the composition, on a w/v basis. In certain further embodiments, the range may be from at least 10% of paraxanthine to 90% and 90% of chlorogenic acid to 10%, respectively.

In further embodiments, paraxanthine and chlorogenic acid are present at a ratio of from 1:4 to about 1:30. In still further embodiments, paraxanthine and chlorogenic acid are present at a ratio from about 1:4 to about 1:10.

In certain embodiments, 1-methylxanthine and chlorogenic acid are present in about equal amounts. In these embodiments, 1-methylxanthine and chlorogenic acid each comprise about 50% of the combined weight of 1-methylxanthine and chlorogenic acid within the composition, on a w/v basis. In certain further embodiments, the range may be from at least 10% of 1-methylxanthine to 90% and 90% of chlorogenic acid to 10%, respectively.

In further embodiments, 1-methylxanthine and chlorogenic acid are present at a ratio of from 1:4 to about 1:30. In still further embodiments, 1-methylxanthine and chlorogenic acid are present at a ratio from about 1:4 to about 1:10.

In certain embodiments, chlorogenic acid is administered to the subject at dose ranging from about 100-150 mg/kg bodyweight of the subject.

In certain embodiments, the composition is formulated such that a dose contains paraxanthine ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein).

In certain embodiments, the composition is formulated such that a dose contains chlorogenic acid ranging from about 500 to about 13,500 mg (e.g., about 500 mg, about 1000 mg, about 1,500 mg, about 2,000 mg, about 2,500 mg, about 3,000 mg, about 3,500 mg, about 4,0000 mg, about 4,500 mg, about 5,000 mg, about 7,500 mg, 10,000, about 13,500 mg, and the like, or any range or value therein).

Depending upon the subject to be treated and the route of administration, the compounds of the invention may be administered at varying doses. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses.

In certain embodiments, the composition is formulated such that a dose contains paraxanthine each ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) and chlorogenic acid ranging from 400 to about 3000 mg (e.g., about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg and the like, or any range or value therein).

In certain embodiments, the composition is formulated such that each dose contains 1-methylxanthine ranging from about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) and chlorogenic acid ranging from 400 to about 3000 mg (e.g., about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg and the like, or any range or value therein).

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, an energy drink, a sublingual, a gummy, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods. As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods. By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Methods of Use

In certain embodiments, paraxanthine may be combined with chlorogenic acid, and in certain embodiments, one or more other chemical compounds (e.g. other active ingredients), to provide a plurality of positive effects in a subject. By altering the dosage of paraxanthine and/or chemical compounds it is combined with, various physiological effects may be selected for. The compositions may provide primarily a single benefit or may provide multiple benefits simultaneously. Depending upon the subject to be treated and the route of administration, the compounds of the invention may be administered at varying doses. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses.

Advantageously, compositions of the present disclosure may be administered in single doses, e.g. once daily or more seldom, or in a total daily dosage administered in divided doses of two, three or four times daily. In certain embodiments, the composition is administered as needed (e.g., when the subject is in need of enhance energy, athletic or cognitive performance or the like).

### Athletic Performance

Further disclosed herein is a method for enhancing performance or energy in subject, comprising administering to the subject a composition disclosed herein. As used herein the term “enhancing performance” is intended to mean any improvement in performance. Performance can be assessed in any manner. Certain enhancements are readily measured. For example, in a timed-event, an improved time can assess an enhanced performance. Certain performance enhancing properties can be judged subjectively by the athlete or performer or an observer. In these instances, an enhanced performance means that the performance was perceived subjectively to be improved, magnified, faster, better and the like. In certain embodiments, the disclosed methods are used to enhance athletic performance. “Athletic performance” refers to any professional or recreational activity wherein the performer, for example an athlete, exerts a physical act, such as running, swimming, golf, bowling, archery, football, baseball, basketball, soccer, hiking, cycling, dancing and the like. In certain athletic performance is improved through in improvement of endurance in the subject. In other words, administration of the disclosed compositions improves a subject's level of endurance, thereby enhancing the subject's athletic performance. In further embodiments, administration of the composition to the subject increases cognitive performance which thereby improves athletic performance.

In certain embodiments, upon administration of the composition, the subject experiences improvement of at least one of mood, energy, focus, concentration or sexual desire or a reduction of at least one of anxiety, fatigue, perception of effort or perception of pain.

In further embodiments, upon continued administration to the subject, the composition does not create dependence in the subject and/or withdrawal effect in the subject when continued use is ceased.

Further disclosed herein is a method of increasing athletic endurance in a subject comprising administering to the subject a composition disclosed herein. In certain implementations, the composition administered to the subject comprises paraxanthine and chlorogenic acid. In exemplary implementations, the administration of paraxanthine and chlorogenic acid produce a synergistic increase athletic endurance in the subject, relative to the administration of paraxanthine or chlorogenic acid alone.

Further disclosed herein is a method of increasing athletic endurance in a subject comprising administering to the subject a composition disclosed herein. In certain implementations, the composition administered to the subject comprises paraxanthine and chlorogenic acid. In exemplary implementations, the administration of paraxanthine and chlorogenic acid produce a synergistic increase athletic endurance in the subject, relative to the administration of paraxanthine or chlorogenic alone.

According to further embodiments, administration of the disclosed composition to the subject increases the subject's perceived level of energy. In exemplary implementations, the subject experiences an increase in energy of at least about 5 percent. According to certain embodiments, the composition administered further comprises (in addition to paraxanthine and/or, 1-methylxanthine and/or chlorogenic acid) at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, huperzine A, theacrine, methylliberine, B12, sulbutiamine, magnolia bark, ketones, MCTs, omega 3's, lutein, zeaxanthin, and n-acetyl-tyrosine, acetyl-1-carnitine and/or combinations thereof.

In certain embodiments, the subject's perceived level of energy is increased by between about 2% and about 50%. In further embodiments, the subject's perceived level of energy is increased by between about 5% and about 30%. In yet further embodiments, the subject's perceived level of energy is increased by between about 10% and about 25%.

### Improved Lipid Profile and Treatment of CVD

Disclosed herein is a method improving serum lipid profile in a subject in a subject, the method comprises administering to the subject an effective amount of a composition comprising from about 2 mg to about 800 mg of paraxanthine and about 25 mg to about 500 mg of chlorogenic acid. In certain aspects, paraxanthine is present in the composition in amount from about 20 mg to about 600 mg. In further aspects, paraxanthine is present in the composition in amount from about 50 mg to about 400 mg.

In another aspect, a method for preventing or treating a cardiovascular condition, which condition includes a pathology of atherosclerotic plaque formation, is provided. The method includes administering to a subject in need thereof a therapeutically effective amount of a composition comprising from about 2 mg to about 800 mg of paraxanthine and about 25 mg to about 500 mg of chlorogenic acid. The cardiovascular condition includes, for example, coronary artery disease, coronary microvascular disease, stroke, carotid artery disease, peripheral arterial disease, and chronic kidney disease. The method can include further slowing down the progression of atherosclerotic plaque formation. The method can further include administering one or more additional therapeutic agent to the subject for the prevention or treatment of the cardiovascular condition.

In another aspect, a method for treating metabolic syndrome is provided. The method includes administering to a subject in need thereof a therapeutically effective amount of a composition comprising from about 2 mg to about 800 mg of paraxanthine and about 25 mg to about 500 mg of chlorogenic acid. The method can further include reducing one or more risk factors associated with metabolic syndrome, including one or more of abdominal obesity, hyperglycemia, dyslipidemia, and hypertension. The method can further include administering one or more additional agent to the subject for the prevention or treatment of metabolic syndrome.

In another aspect, a method for delaying or slowing down the progression of atherosclerosis is provided. The method includes administering to a subject in need thereof a therapeutically effective amount of a composition comprising from about 2 mg to about 800 mg of paraxanthine and about 25 mg to about 500 mg of chlorogenic acid.

### Weight Loss/Body Composition

Paraxanthine exhibits a wide variety of effects depending on dosage. The presence of other ingredients may also modulate its effects. It may be used to improve lipolysis, thermogenesis, and/or decreased appetite. In one embodiment, paraxanthine may be used to promote weight loss by reducing appetite, act as an antioxidant and as an anti-inflammatory. Paraxanthine may be used transdermal to enhance one or more of these effects. In another embodiment, a dietary supplement comprising about 2 mg to about 800 mg paraxanthine and about 25 mg to about 500 mg of chlorogenic acid, is provided. In another embodiment, a nutritional supplement to support weight loss and/or fat loss through lipolysis for improving the body composition is provided. It is therefore an object of the present disclosure to provide compositions including paraxanthine capable of imparting a plurality of positive effects on a subjects body composition. It is another object of the present disclosure to provide congeners, derivatives and iterations of paraxanthine and synthetic chemical equivalents of paraxanthine. It is another object of the present disclosure to provide agglomerated paraxanthine, paraxanthine salts, microencapsulated, liposomal or esterified paraxanthine. It is another object of the present disclosure to provide paraxanthine combined with glycerides, propylene glycol, polyethylene glycol (PEG), lauroyl macrogol, lauroyl macrogol derivatives and co-crystallization products of paraxanthine.

Further disclosed herein is a method for promoting weight loss and/or weight management in subject by providing the subject with a composition comprising about 2 mg to about 800 mg of paraxanthine. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses In certain aspects, paraxanthine is present in the composition in amount from about 20 mg to about 600 mg. In further aspects, paraxanthine is present in the composition in amount from about 50 mg to about 400 mg.

According to certain embodiments, weight loss is promoted through inducing thermogenesis in the subject. According to exemplary implementations of these embodiments, the composition may also include one or more compounds selected from: caffeine, green tea, capsaicin, garcinia cambogia, yohimbine, catechols, EGCG, catechins, and proanthocyanidins and octacosanol and bitter orange.

According to further embodiments, weight loss is promoted through suppression of appetite in the subject. In exemplary implementations of these embodiments, the composition further may include one or more compounds selected from: fenugreek, glucomannan, gymnema sylvestre, 5-HTP, Caralluma fimbriata, green tea extract, Conjugated linoleic acid, Garcinia cambogia, and Yerba mate.

According to still further embodiments, weight loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides, catechols, EGCG, catechins, and proanthocyanidins and octacosanol and octacosanol.

According to certain implementations, the disclosed method further comprises restricting calorie intake of the subject. In exemplary implementations, the amount of weight loss in the subject is greater than that for a subject with an equivalent calorie restriction that has not been provided the composition. According to further implementations, the ratio of fat loss to muscle loss in the subject the subject is greater than that for a subject with an equivalent caloric restriction that has not been provided the composition.

### Inhibition of Inflammation

Further disclosed herein is a method of inhibiting inflammation in a subject in need thereof comprising administering to the subject a composition comprising about 2 mg to about 800 mg of paraxanthine and about 25 mg to about 500 mg of chlorogenic acid. In certain aspects, paraxanthine is present in the composition in amount from about 20 mg to about 600 mg. In further aspects, paraxanthine is present in the composition in amount from about 50 mg to about 400 mg.

In certain embodiments, the subject is at risk of developing inflammatory disease or condition. In further embodiments, the subject has been diagnosed with an inflammatory disease or condition. In exemplary implementations, the subject has been diagnosed with diabetes, Crohn's disease, rheumatoid arthritis, fibromyalgia, systemic lupus erythematosus, glomerulonephritis, scleroderma, or multiple sclerosis.

In still further embodiments, the one or more additional ingredient is selected from omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothionecine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum.

Further disclosed herein are method for improving joint health by administering to a subject in need thereof a composition comprising paraxanthine. In certain embodiments, the composition further comprises dileucine.

According to certain embodiments, improving joint health comprises alleviating or reducing the severity of at least one symptom of osteoarthritis, such as, for example, pain, stiffness, tenderness, reduced flexibility, grating sensation, bone spurs, swelling, or any combination thereof. Thus, in some embodiments, there is provided a method of reducing the severity of at least one symptom of osteoarthritis in a subject with osteoarthritis, comprising administering to the subject an herbal composition of the present disclosure.

In some embodiments of the fourth aspect, the improvement in joint health may be measured by changes in baseline versus end point scores in the 30 second chair stand test (30SCST). Thus, in some embodiments, provided is a method of improving the 30 second chair stand test (30SCST) score in a subject with an inflammatory joint condition comprising administering to the subject a composition of the present disclosure for at least 30 days, at least 60 days, at least 90 days, at least 120 days, or longer. In some embodiments, the improvement in the 30SCST at 120 days is at least 8%, at least 10%, at least 13%, at least 15%, at least 16%, or at least about 18%.

In some embodiments of the fourth aspect, the improvement in joint health may be measured by changes in baseline versus end point range of motion in knee flexion. Thus, in some embodiments, there is provided a method of improving the range of motion in knee flexion in a subject with an inflammatory joint condition comprising administering to the subject a composition of the present disclosure for at least 30 days, at least 60 days, at least 90 days, at least 120 days, or longer. In some embodiments, the range of motion in knee flexion at 120 days is improved by at least 4%, at least 5%, at least 6%, or at least 7%.

As described herein, an effective amount of the composition can be administered to the subject once per day. In some embodiments, an effective amount of the composition or can be administered to a subject as multiple doses per day, for example twice per day or more frequently, three times per day or more frequently, or four times per day or more frequently. In some embodiments, an effective amount of the composition can be administered to a subject once per week or more frequently, twice per week or more frequently, three times per week or more frequently, four times per week or more frequently, five times per week or more frequently, or six times per week or more frequently.

### Cognitive Function

Disclosed herein is a method of enhancing cognitive function in a subject comprising administering to the subject a composition disclosed herein. In certain embodiments, improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

In certain embodiments, administration of the disclosed composition increases working memory.

In further embodiments, administration of the disclosed composition increases attention.

According to certain embodiments, composition of the instantly disclosed methods to enhance cognitive function further comprise, N-acetyl-tyrosine, taurine, huperzine A, acetyl-1-carnitine, CDP choline, Alpha GPC, choline bitrate, choline citrate, B12, caffeine, methyllliberine, theacrine, paraxanthine, theobromine, ashwagandha, rhodiola, lutein, zeaxanthin, fish oil, creatine, ginseng, lions mane, niacin, cordyceps, theanine, B-vitamins, GABA, sulbutiamine, vinpocetine, adenosine triphosphate, inositol, enhanced arginine silicate, nitrates, electrolytes, hesperidin and derivatives of hesperidin and/or bacopa.

In certain embodiments, the subject has experience age-related cognitive decline. In exemplary implementations, administration of the composition to the subject increases the level BDNF in the subject. According to certain embodiments, administration of the composition to the subject increases brain derived neurotrophic factor (BDNF) levels in the subject. In exemplary implementations, BDNF levels are increased by from about 5% to about 40%. In further embodiments, BDNF levels are increased by at least about 15%. In further embodiments, administration of the composition to the subject increases other neurotrophic factors such as neuronal growth factor (NGF).

### Methods of Treatment

Further disclosed herein is a method of treating a condition in a subject in need thereof by administering to the subject a composition disclosed herein. In certain embodiments, the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and dementia.

Further disclosed herein is a method for treating a mood disorder by administering to a subject in need thereof a composition disclosed herein. In certain embodiments, the mood disorder is selected from clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, comorbid depression, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein.

In certain embodiments, the mood disorder is depression. In exemplary implementations, subject has been diagnosed with depression or is at risk of depression.

Further disclosed herein is a method for treating an anxiety disorder in a subject in need thereof by administering to a subject in need thereof a composition disclosed herein. In certain embodiments, the anxiety disorder is selected from: generalized anxiety disorder, panic disorder, obsessive compulsive disorder, phobia, post-traumatic stress disorder). As will be appreciated by those skilled in the art, anxiety disorder is a blanket term covering several different forms of abnormal and pathological fear and anxiety.

According to certain embodiments, the composition is administered in a therapeutically effective amount. In further embodiments, the composition is administered in a prophylactically effective amount.

In certain embodiments, the composition used in the method of treating a mood disorder or anxiety disorder further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino)ethanol (DMAE), DMAE bitartrate, magnolia bark, theanine, phosphatidylserine, ashwagandha, rhodiola, macuna, Sceletium tortuosa, 5-HTP, tryptophan, saffron, Vitamin D, SAMe, lions mane and/or huperzine A.

Further disclosed herein is a method for treating or preventing age-related cognitive decline in a subject in need thereof, comprising administering to the subject an effective amount of a composition disclosed herein. In certain embodiments, administration of the composition increases one or more of attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition. In certain embodiments, administration of the composition to the subject enhances working memory in the subject.

In certain embodiments, administration of the composition to the subject enhances BDNF expression, glutathione levels, and/or dopaminergic tone, serotonergic tone, cholinergic tone, and/or reduces stress induced cortisol levels.

According to certain embodiments, the composition disclosed herein are used in the treatment of one or more medical conditions in a subject in need thereof. In certain implementations, the disclosed composition is administered to a subject suffering from narcolepsy, sleep apnea, and shift work sleep disorder, insomnia epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and/or dementia.

In certain aspects, the disclosed compositions are a neuroprotective agent. In certain embodiments, administration of the disclosed compositions to a subject in need thereof is neuroprotective. In exemplary aspects of these embodiments, this neuroprotection is in the form of protecting against dopaminergic cell death.

According to further embodiments, disclosed compositions are useful for the treatment of geriatric depression. In exemplary embodiments, the compositions are effective in treating subjects suffering from geriatric depression an essential, vascular or traumatic origin. And of the mental decay in the elderly.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In another embodiment, a combination of paraxanthine and chlorogenic acid may be used at lower dosage levels and/or in conjunction with compounds that modulate or antagonize its activity. Such compositions may induce an improved endurance performance, mood, vigor, lipolysis, energy expenditure, exercise performance, and/or decreased appetite.

An advantage of using the disclosed compositions is the reduced likelihood that a person develops a tolerance to chemical compositions. That is, a person may not become desensitized to the effects induced. According to certain aspects, the disclosed combination of paraxanthine and chlorogenic acid containing compositions has at least the following distinct advantages over the administration of compositions containing comparable doses of caffeine. The combination of paraxanthine and chlorogenic acid has substantially lower toxicity. The combination of paraxanthine and chlorogenic acid has greater stability (e.g. does not lose potency over time to the same extent as caffeine).

A composition containing a combination of paraxanthine and chlorogenic acid is a more potent wake-promoting agent (in certain embodiments, via adenosine receptor antagonism).

Further, compositions containing a combination of paraxanthine and chlorogenic acid enhance striatal dopaminergic tone. Still further, the combination of paraxanthine and chlorogenic acid does not produce sleep rebound. Further, a combination of paraxanthine and chlorogenic acid does not produce withdrawal effects upon cessation of use, as frequently occurs with caffeine. Yet further, a combination of paraxanthine and chlorogenic acid does not enhance anxiety. Still further, a combination of paraxanthine and chlorogenic acid is less bitter than caffeine. Even further, a combination of paraxanthine and chlorogenic acid is effective for a larger portion of the population than caffeine. In another embodiment, a combination of paraxanthine and chlorogenic acid may be used at higher dosage levels and/or with synergistic compounds.

Further disclosed herein is a method for improving sleep in a subject by administering a composition disclosed herein. In certain embodiments, the subject suffers from insomnia. In certain embodiments, the administration of 1-methylxanthine and chlorogenic acid produce a synergistic improvement of sleep in the subject, relative to the administration of 1-methylxanthine or chlorogenic acid alone. In further embodiments, the administration of paraxanthine and chlorogenic acid produce a synergistic improvement of sleep in the subject, relative to the administration of paraxanthine or chlorogenic acid alone.

These compositions may increase a person's basal/resting metabolic rate, increase thermogenesis, decrease appetite, enhance cognitive performance, increase alpha wave brain activity, and/or induce euphoria. Without being bound by theory, the inventors believe that at higher dosage levels, a composition containing a combination of paraxanthine and chlorogenic acid may be noradrenergic and dopaminergic and may exhibit increased adenosine receptor inhibition.

In another embodiment, combination of paraxanthine and chlorogenic acid may be used as a topical agent for incorporation into body creams or lotions to produce a cream or lotion for lightening skin, firming skin, and/or improving skin elasticity. A topical agent containing a combination of paraxanthine and chlorogenic acid may also be used to promote localized transdermal fat loss. Such a composition may also be used in a cream or lotion to promote localized enhanced metabolism and/or enhanced thermogenesis.

According to further embodiments, paraxanthine and chlorogenic acid may be combined with one or more of analgesics and/or anti-inflammatory agents. In exemplary implementations, paraxanthine and chlorogenic acid are combined with ibuprofen, salicylic acid, anti-inflammatory agents, salicin, fish oil (omega-3 fatty acids and specialized, small lipid pro-resolving derivatives), tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate and/or triterpenoids.

The dosage of combination of paraxanthine and chlorogenic acid may range from about 100 mg to about 3000 mg. In another embodiment, the range may be from about 500 mg to about 2500 mg. In further embodiments, the combined dose of paraxanthine and chlorogenic acid is about 600 mg. In another embodiment, the range may be from at least 10% of paraxanthine to 90% and 90% of chlorogenic acid to 10%, respectively.

In certain embodiments, the composition comprises paraxanthine and chlorogenic acid at a ratio of about 1:5. In certain embodiments, the amount of paraxanthine provided is about 2 mg to about 800 mg and the amount of chlorogenic acid provided is about 500 mg to about 2000 mg.

In exemplary implementations, the composition is administered at a dose of about 100 mg paraxanthine and about 500 mg chlorogenic acid.

In another embodiment, a combination of paraxanthine and chlorogenic acid is combined with one or more bioavailability enhancers. In exemplary embodiments, bioavailability enhancers include, but are not limited to: bioperine, piperine, black pepper, bergamottin, dihydroxybergamottin (CYP3A4 inhibitors), flavonoids (including hesperidin, naringin, tangeritin, quercetin and nobiletin both in isolation and in combination), pterostilbenes, fisctin, nanoencapsulation, microencapsulation, liposomes and/or phytosomes. The enhancers that are combined with combination of paraxanthine and chlorogenic acid may depend on which qualities of combination of paraxanthine and chlorogenic acid are desired for a particular use.

In another embodiment, the combination of paraxanthine and chlorogenic acid may be administered using one or more delivery methods, including, for example transdermal patches and/or creams, ready to mix powders, intravenous methods, capsules, tablets, liquid (including liquids for mixing with other beverages), softgels, shot format, and/or cosmetic applications including soaps, lotions and shampoos. combination of paraxanthine and chlorogenic acid's anti-inflammatory qualities may be desired for a variety of topical applications.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

According to certain embodiments, various aspects and embodiments of the present invention are defined by the following numbered clauses:

1. A composition comprising: about 50 mg to about 400 mg of paraxanthine and at least about 25 mg chlorogenic acid.
2. The composition of clause 1, wherein chlorogenic acid is present in the composition in amount from about 25 mg to about 500 mg.
3. The composition of clause 1, wherein paraxanthine is present in the composition in amount from about 200 mg to about 400 mg.
4. A composition comprising: about 50 mg to about 400 mg of 1-methylxanthine and at least about 25 mg chlorogenic acid.
5. A method of treating a condition in a subject in need thereof, comprising administering to the subject the composition of any of clause 1-4.
6. The method of clause 5, wherein the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), concussion, Parkinson's disease, Alzheimer's, and dementia.
7. The method of clause 6, wherein the condition is a mood disorder.
8. The method of clause 7, wherein the mood disorder is depression.
9. The method of clause 8, wherein the subject has been diagnosed with depression or is at risk of depression.
10. The method of clause 7, wherein the composition further comprises at least one ingredient selected from the group consisting of L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum and/or combinations thereof, caffeine, theobromine, naringin, hesperidin, 2-(dimethylamino) ethanol (DMAE), DMAE bitartrate, magnolia bark, theanine, phosphatidylserine, ashwagandha, rhodiola, n-acetyl-tyrosine, macuna, sceletium tortuosa, 5-HTP, tryptophan, saffron, Vitamin D, SAMe, lions mane and/or huperzine A.
11. A method of enhancing attention in a subject in need thereof comprising administering the composition of any of clauses 1-4.
12. A method of improving working memory in a subject in need thereof comprising administering a composition to the subject comprising the composition of any of clauses 1-4.
13. A method of improving cognitive performance in a subject comprising administering the composition of any of clause 1-4.
14. The method of clause 13, wherein improved cognitive function is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.
15. A method of preventing or treating TBI in a subject in need thereof the composition of any of clauses 1-4.
16. The method of clause 15, wherein administration of the composition to the subject enhances expression of Brain Derived Neurotrophic Factor (BDNF), relative to the brain of subject who has not been administered the composition.
17. The method of clauses 15-16, wherein administration of the composition prevents the occurrence of TBI when administered to the subject prior to injury.
18. A method for enhancing neuroplasticity in a subject in need thereof, comprising administering to the subject the composition of any of clauses 1-4.
19. The method of clause 18, wherein the amount for paraxanthine and/or 1-methylxanthine is administered to the subject is from about 500 mg to about 400 mg and wherein the amount for chlorogenic acid administered to the subject is from about 50 mg to about 500 mg and wherein the administration of paraxanthine and/or 1-methylxanthine and chlorogenic acid produce a synergistic enhancement of neuroplasticity in the subject, relative to the administration of paraxanthine and/or 1-methylxanine or chlorogenic acid alone.
20. A method for improving digestive health in a subject in need thereof, comprising administering to the subject the composition of any of clauses 1-4.
21. The method of clause 20, wherein digestive health is improved through enhancing microbiota in the subject.
22. The method of clause 21, wherein the amount for paraxanthine administered to the subject is from about 500 mg to about 400 mg and wherein the amount for chlorogenic acid administered to the subject is from about 50 mg to about 500 mg and wherein the administration of paraxanthine and chlorogenic acid produce a synergistic improvement of digestive health in the subject, relative to the administration of paraxanthine or chlorogenic acid alone
23. A method for attenuating hypertension in a subject in need thereof, comprising administering to the subject the composition of any of clauses 1-4.
24. The method of clause 23, wherein the amount for paraxanthine administered to the subject is from about 500 mg to about 400 mg and wherein the amount for chlorogenic acid administered to the subject is from about 50 mg to about 500 mg and wherein the administration of paraxanthine and chlorogenic acid produce a synergistic attenuation of hypertension in the subject, relative to the administration of paraxanthine or chlorogenic acid alone.
25. A method of enhancing energy or mood in a subject comprising administering to the subject a composition comprising an effective amount of paraxanthine and chlorogenic acid, wherein the amount for paraxanthine administered to the subject is from about 500 mg to about 400 mg and wherein the amount for chlorogenic acid administered to the subject is from about 50 mg to about 500 mg and wherein the administration of paraxanthine and chlorogenic acid produce a synergistic enhancement in energy and/or mood in the subject, relative to the administration of paraxanthine or chlorogenic acid alone.
26. A method of modulating cellular energy homeostasis comprising administering to a subject the composition of any of clauses 1-4.
27. The method of clause 26, wherein administration of the composition inhibits 5′ AMP-activated protein kinase and/or CD38 in the subject.
28. The method of clause 26, wherein administration of the composition enhances NAD levels in the subject.
29. The method of clause 26, wherein administration of the composition reduces insufficient cellular energy status in the subject.
30. The method of any of clauses 26-29, wherein the amount for paraxanthine administered to the subject is from about 500 mg to about 400 mg and wherein the amount for chlorogenic acid administered to the subject is from about 50 mg to about 500 mg and wherein the administration of paraxanthine and chlorogenic acid produce a synergistic modulation of cellular energy homeostasis in the subject, relative to the administration of paraxanthine or chlorogenic acid alone.
31. A method of modulating blood glucose levels in a subject comprising administering to a subject the composition of any of clauses 1-4.
32. The method of clause 31, wherein administration of the composition to the subject reduces advanced glycation end products in the subject.
33. The method of clause 31, wherein administration of the composition to the subject reduces glycation in the subject.
34. The method of any preceding clause, further comprising administration to the subject a composition comprising metabolites of paraxanthine and/or chlorogenic acid.
35. The method of any preceding clause, wherein the composition administered to the subject is substantially free of caffeine.
36. A composition comprising: about 50 mg to about 400 mg of paraxanthine or 1-methylxanthine and at least about 25 mg chlorogenic acid.
37. The composition of clause 36, wherein chlorogenic acid is present in the composition in an amount from about 25 mg to about 500 mg.
38. The composition of clause 36, the composition comprises paraxanthine in an amount from about 200 mg to about 400 mg.
39. The composition of clause 36, the composition comprises 1-methylxantine in an amount from about 200 mg to about 400 mg.
40. A method for improving cognitive performance in a subject comprising administering a composition comprising: about 50 mg to about 400 mg of paraxanthine or 1-methylxanthine and at least about 25 mg chlorogenic acid.
41. The method of clause 40, wherein chlorogenic acid is present in the composition in an amount from about 25 mg to about 500 mg.
42. The method of clause 40, wherein the composition comprises paraxanthine in an amount from about 200 mg to about 400 mg.
43. The method of clause 42, wherein the administration of paraxanthine and chlorogenic acid produce a synergistic enhancement of cognitive function in the subject, relative to the administration of paraxanthine or chlorogenic acid alone.
44. The method of clause 40, wherein the composition comprises 1-methylxantine in an amount from about 200 mg to about 400 mg.
45. The method of clause 44, wherein the administration of 1-methylxanthine and chlorogenic acid produce a synergistic enhancement of cognitive function in the subject, relative to the administration of 1-methylxanthine or chlorogenic acid alone.
46. The method of clause 40, wherein improved cognitive performance is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.
47. The method of clause 46, wherein improved cognitive performance is measured by improved working memory.
48. The method of clause 40, wherein administration of the composition to the subject enhances BDNF expression, glutathione levels, and/or dopaminergic tone, serotonergic tone, cholinergic tone, and/or reduces stress induced cortisol levels.
49. A method for improving sleep in a subject, comprising administering a composition comprising: about 50 mg to about 400 mg of paraxanthine or 1-methylxanthine and at least about 25 mg chlorogenic acid.
50. The method of clause 49, wherein chlorogenic acid is present in the composition in an amount from about 25 mg to about 500 mg.
51. The method of clause 49, wherein the composition comprises paraxanthine in an amount from about 200 mg to about 400 mg.
52. The method of clause 51, wherein the administration of paraxanthine and chlorogenic acid produce a synergistic improvement of sleep in the subject, relative to the administration of paraxanthine or chlorogenic acid alone.
53. The method of clause 49, wherein the composition comprises 1-methylxantine in an amount from about 200 mg to about 400 mg.
54. The method of clause 53, wherein the administration of 1-methylxanthine and chlorogenic acid produce a synergistic improvement of sleep in the subject, relative to the administration of 1-methylxanthine or chlorogenic acid alone.
55. The method of clause 49, wherein the subject suffers from insomnia.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1—Evaluation of Various Implementations on Pentobarbital Induced Sleep

While multiple implementations are disclosed, still other implementations of the disclosure will become apparent to those skilled in the art from the following detailed description, which shows and describes illustrative implementations of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

The present example was conducted to evaluate the efficacy of test substances on Pentobarbital Sodium induced sleep activity in experimental mice. Table 1 below shows information about the mice.

TABLE 1
Mice Data.

| Strain | Swiss Albino Mice | |
|---|---|---|
| Sex | Male | |
| Age | 8-10 | Weeks |
| Body Weight | 22-25 | rams |

The mice were randomized into nine groups containing eight mice in each group. These groups were the Normal Control group, which did not receive doses of Pentobarbital sodium or any other test substance; the Pathological Control group, which received doses of Pentobarbital sodium and scopolamine; the PX group, which received doses of Pentobarbital sodium and Paraxanthine; the CA group, which received doses of Pentobarbital sodium and Chlorogenic acid; the 1-MX group, which received doses of Pentobarbital sodium and 1-Methylxanthine; the PX+CA group, which received doses of Pentobarbital sodium, Paraxanthine, and Chlorogenic acid; and the 1-MX+CA group, which received doses of Pentobarbital sodium, 1-Methylxanthine, and Chlorogenic acid. The dose information can be found below in Table 2.

On the day of experiment, mice from different groups were treated with the control and test substances respectively. After thirty minutes, all groups of mice, except for the Normal Control group were, administered with 40 mg/kg body doses of Pentobarbital sodium to induce sleep. Administration of the test substances was done via intraperitoneal injection. The onset time of sleep was recorded for all the mice. After induction of sleep, mice were placed in the inverted position and when sedation was over, time point where the mice return to its normal posture was noted. The time interval between dosing and start of sleep was recorded as latency time. Table 2 shows the data recorded from the example.

TABLE 2
Effects on Pentobarbital Induced Sleep.

| | | Improve- | | Improve- |
|---|---|---|---|---|
| | Onset | ment | Duration | ment |
| Effect on Pentobarbital | time of | over | of | over |
| Induced Sleep | sleep | P-Control | sleep | P-Control |
| Activity in Mice | (min) | [%] | (min) | [%] |
| Normal Control | 0 | | 0.00 | |
| Pathological Control | 50.5 | | 27.88 | |
| (Scopolamine) | | | | |
| 50 mg HED Paraxanthine | 41.5 | −18 | 66.13 | 137 |
| (PX) | | | | |
| 25 mg HED Chlorogenic | 40.5 | −20 | 74.88 | 169 |
| acid (CA) | | | | |
| 50 mg HED 1- | 42.25 | −16 | 57.75 | 107 |
| Methylxanthine (1-MX) | | | | |
| 50 mg HED PX + 25 mg | 28 | −45 | 89.5 | 221 |
| HED CA | | | | |
| 50 mg HED 1-MX + 25 mg | 35.88 | −29 | 79.25 | 184 |
| HED CA | | | | |

### Example 2—Evaluation of Various Implementations on Cognitive Enhancing Effect Based on Plus-Maze Model

The example was to evaluate the effect of the test substances on behavioral activity/memory enhancing activity in experimental rats. Table 3 presents data about the rats selected for the example.

TABLE 3
Rat Data.

| Strain | Swiss Albino Wistar | |
|---|---|---|
| Sex | Male | |
| Age | 8-10 | Weeks |
| Body Weight | 120-200 | grams |

The rats were randomized into nine groups containing eight rats in each group. These groups were the Normal Control group, which did not receive doses of any test substance; the Pathological Control group, which received doses of Scopolamine only; the PX group, which received doses of scopolamine and Paraxanthine; the CA group, which received doses of Scopolamine and Chlorogenic acid; the 1-MX group, which received doses of scopolamine and 1-Methylxanthine; the PX+CA group, which received doses of Scopolamine, Paraxanthine, and Chlorogenic acid; and the 1-MX+CA group, which received doses of Scopolamine, 1-Methylxanthine, and Chlorogenic acid. The rats were treated with test substances daily for ten consecutive days.

On the tenth day test, test substances were administered orally thirty minutes before a 1 mg/kg body weight Scopolamine injection to induce amnesia in rats to all groups except control group. Administration of the Scopolamine injections were done via intraperitoneal injection. This was followed by behavioral test using Elevated Plus Maze apparatus to measure the long-term memory enhancing activity. Table 4 shows the data recorded from the example.

TABLE 4
Effects on Cognitive Enhancing Effect - Elevated Plus Maze.

| | | Improvement |
|---|---|---|
| Effect of test substance on rat escape | Latency | over P-Control |
| latency (sec) | [sec] | [%] |
| Normal Control | 16.36 | |
| Pathological Control (Scopolamine) | 50.41 | |
| 50 mg HED Paraxanthine (PX) | 36.66 | 38 |
| 25 mg HED Chlorogenic acid (CA) | 30 | 68 |
| 50 mg HED 1-Methylxanthine | 33.89 | 49 |
| (1-MX) | | |
| 50 mg HED PX + 25 mg HED CA | 19.59 | 157 |
| 50 mg HED 1-MX + 25 mg HED CA | 28.3 | 78 |

Several brain biomarkers were also measured in the rats. Tables 5 and 6 present this data.

TABLE 5
Brain Biomarker Concentration and Cognitive Improvement.

| | | Improvement | | Improvement | | Improvement |
|---|---|---|---|---|---|---|
| Effect of test substance on brain | Achetylcholine | over P-Control | Dopamine | over P-Control | Glutathione | over P-Control |
| biomarkers | (U/ml) | [%] | (ng/L) | [%] | (μg/mL) | [%] |
| Normal Control | 58.73 | | 25.51 | | 26.62 | |
| Pathological Control (Scopolamine) | 21.54 | | 11.6 | | 13.58 | |
| 50 mg HED Paraxanthine (PX) | 32.04 | 49 | 15.06 | 30 | 19.57 | 44 |
| 25 mg HED Chlorogenic acid (CA) | 33.17 | 54 | 15.58 | 34 | 19.89 | 46 |
| 50 mg HED 1-Methylxanthine (1-MX) | 28.97 | 34 | 14.51 | 25 | 17.94 | 32 |
| 50 mg HED PX + 25 mg HED CA | 38.23 | 77 | 16.73 | 44 | 23.98 | 77 |
| 50 mg HED 1-MX + 25 mg HED CA | 34.15 | 59 | 16.08 | 39 | 22.13 | 63 |

TABLE 6
Brain Biomarker Concentration and Cognitive Improvement.

| | | Improvement | | Improvement | | Improvement | | Improvement |
|---|---|---|---|---|---|---|---|---|
| Effect of test substance on brain | Catalse | over P-Control | BDNF | over P-Control | Serotonin | over P-Control | Cortisol | over P-Control |
| biomarkers | (U/mL) | [%] | (pg/mL) | [%] | (ng/ml) | [%] | (ng/ml) | [%] |
| Normal Control | 176.95 | | 26.17 | | 23.76 | | 51.24 | |
| Pathological Control (Scopolamine) | 120.83 | | 13.19 | | 47.52 | | 90.36 | |
| 50 mg HED Paraxanthine (PX) | 159.83 | 32 | 17.72 | 34 | 38.03 | −20 | 73.99 | −18 |
| 25 mg HED Chlorogenic acid (CA) | 161.91 | 34 | 18.05 | 37 | 38.43 | −19 | 68.51 | −24 |
| 50 mg HED 1-Methylxanthine (1-MX) | 155.96 | 29 | 17.29 | 31 | 41.63 | −12 | 79.4 | −12 |
| 50 mg HED PX + 25 mg HED CA | 171.01 | 42 | 23.97 | 82 | 30.82 | −35 | 67 | −26 |
| 50 mg HED 1-MX + 25 mg HED CA | 165.51 | 37 | 22.12 | 68 | 33.76 | −29 | 67.4 | −25 |

### Example 3—Evaluation of Various Implementations on Cognitive Enhancing Effect Based on Morris Water Maze

The example was to evaluate the effect of the test substances on behavioral activity/memory enhancing activity in experimental rats. Table 7 presents data about the rats selected for the example.

TABLE 7
Rat Data.

| Strain | Swiss Albino Wistar | |
|---|---|---|
| Sex | Male | |
| Age | 8-10 | Weeks |
| Body Weight | 120-200 | grams |

The rats were randomized into nine groups containing eight rats in each group. These groups were the Normal Control group, which did not receive doses of any test substance; the Pathological Control group, which received doses of Scopolamine only; the PX group, which received doses of scopolamine and Paraxanthine; the CA group, which received doses of Scopolamine and Chlorogenic acid; the 1-MX group, which received doses of scopolamine and 1-Methylxanthine; the PX+CA group, which received doses of Scopolamine, Paraxanthine, and Chlorogenic acid; and the 1-MX+CA group, which received doses of Scopolamine, 1-Methylxanthine, and Chlorogenic acid. The rats were treated with test substances daily for ten consecutive days.

On the tenth day test, test substances were administered orally thirty minutes before a 1 mg/kg body weight Scopolamine injection to induce amnesia in rats to all groups except control group. Administration of the Scopolamine injections were done via intraperitoneal injection. This was followed by behavioral test using Morris Water Maze apparatus to measure the long-term memory enhancing activity. Table 8 shows the data recorded from the example.

TABLE 8
Effects on Cognitive Enhancing Effect - Morris Water Maze.

| | | Improvement |
|---|---|---|
| Effect of test substance on rat escape | Latency | over P-Control |
| latency (sec) | [sec] | [%] |
| Normal Control | 14.90 | |
| Pathological Control (Scopolamine) | 48.95 | |
| 50 mg HED Paraxanthine (PX) | 30.46 | 61 |
| 25 mg HED Chlorogenic acid (CA) | 28.54 | 72 |
| 50 mg HED 1-Methylxanthine | 32.43 | 51 |
| (1-MX) | | |
| 50 mg HED PX + 25 mg HED CA | 18.13 | 170 |
| 50 mg HED 1-MX + 25 mg HED CA | 26.84 | 82 |

Several brain biomarkers were also measured in the rats. Tables 9 and 10 present this data.

TABLE 9
Brain Biomarker Concentration and Cognitive Improvement.

| | | Improvement | | Improvement | | Improvement |
|---|---|---|---|---|---|---|
| Effect of test substance on brain | Achetylcholine | over P-Control | Dopamine | over P-Control | Glutathione | over P-Control |
| biomarkers | (U/ml) | [%] | (ng/L) | [%] | (μg/mL) | [%] |
| Normal Control | 57.41 | | 24.66 | | 25.48 | |
| Pathological Control (Scopolamine) | 20.21 | | 10.3 | | 11.97 | |
| 50 mg HED Paraxanthine (PX) | 34.16 | 69 | 14.21 | 38 | 17.47 | 46 |
| 25 mg HED Chlorogenic acid (CA) | 33.03 | 63 | 14.74 | 43 | 16.85 | 41 |
| 50 mg HED 1-Methylxanthine (1-MX) | 30.87 | 53 | 13.66 | 33 | 16.06 | 34 |
| 50 mg HED PX + 25 mg HED CA | 36.9 | 83 | 15.89 | 54 | 22.84 | 91 |
| 50 mg HED 1-MX + 25 mg HED CA | 36.18 | 79 | 14.25 | 38 | 20.99 | 75 |

TABLE 10
Brain Biomarker Concentration and Cognitive Improvement.

| | | Improvement | | Improvement | | Improvement |
|---|---|---|---|---|---|---|
| Effect of test substance on brain | Achetylcholine | over P-Control | Dopamine | over P-Control | Glutathione | over P-Control |
| biomarkers | (U/ml) | [%] | (ng/L) | [%] | (μg/mL) | [%] |
| Normal Control | 57.41 | | 24.66 | | 25.48 | |
| Pathological Control (Scopolamine) | 20.21 | | 10.3 | | 11.97 | |
| 50 mg HED Paraxanthine (PX) | 34.16 | 69 | 14.21 | 38 | 17.47 | 46 |
| 25 mg HED Chlorogenic acid (CA) | 33.03 | 63 | 14.74 | 43 | 16.85 | 41 |
| 50 mg HED 1-Methylxanthine (1-MX) | 30.87 | 53 | 13.66 | 33 | 16.06 | 34 |
| 50 mg HED PX + 25 mg HED CA | 36.9 | 83 | 15.89 | 54 | 22.84 | 91 |
| 50 mg HED 1-MX + 25 mg HED CA | 36.18 | 79 | 14.25 | 38 | 20.99 | 75 |

What is claimed is:

1. A composition comprising: about 50 mg to about 400 mg of paraxanthine or 1-methylxanthine and at least about 25 mg chlorogenic acid.

2. The composition of claim 1, wherein chlorogenic acid is present in the composition in an amount from about 25 mg to about 500 mg.

3. The composition of claim 1, the composition comprises paraxanthine in an amount from about 200 mg to about 400 mg.

4. The composition of claim 1, the composition comprises 1-methylxantine in an amount from about 200 mg to about 400 mg.

5. A method for improving cognitive performance in a subject comprising administering a composition comprising: about 50 mg to about 400 mg of paraxanthine or 1-methylxanthine and at least about 25 mg chlorogenic acid.

6. The method of claim 5, wherein chlorogenic acid is present in the composition in an amount from about 25 mg to about 500 mg.

7. The method of claim 5, wherein the composition comprises paraxanthine in an amount from about 200 mg to about 400 mg.

8. The method of claim 7, wherein the administration of paraxanthine and chlorogenic acid produce a synergistic enhancement of cognitive function in the subject, relative to the administration of paraxanthine or chlorogenic acid alone.

9. The method of claim 5, wherein the composition comprises 1-methylxantine in an amount from about 200 mg to about 400 mg.

10. The method of claim 9, wherein the administration of 1-methylxanthine and chlorogenic acid produce a synergistic enhancement of cognitive function in the subject, relative to the administration of 1-methylxanthine or chlorogenic acid alone.

11. The method of claim 5, wherein improved cognitive performance is measured by an increase in one or more of: attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, speed of processing, reasoning, problem solving and/or social cognition.

12. The method of claim 11, wherein improved cognitive performance is measured by improved working memory.

13. The method of claim 5, wherein administration of the composition to the subject enhances BDNF expression, glutathione levels, and/or dopaminergic tone, serotonergic tone, cholinergic tone, and/or reduces stress induced cortisol levels.

14. A method for improving sleep in a subject, comprising administering a composition comprising: about 50 mg to about 400 mg of paraxanthine or 1-methylxanthine and at least about 25 mg chlorogenic acid.

15. The method of claim 14, wherein chlorogenic acid is present in the composition in an amount from about 25 mg to about 500 mg.

16. The method of claim 114, wherein the composition comprises paraxanthine in an amount from about 200 mg to about 400 mg.

17. The method of claim 16, wherein the administration of paraxanthine and chlorogenic acid produce a synergistic improvement of sleep in the subject, relative to the administration of paraxanthine or chlorogenic acid alone.

18. The method of claim 14, wherein the composition comprises 1-methylxantine in an amount from about 200 mg to about 400 mg.

19. The method of claim 18, wherein the administration of 1-methylxanthine and chlorogenic acid produce a synergistic improvement of sleep in the subject, relative to the administration of 1-methylxanthine or chlorogenic acid alone.

20. The method of claim 14, wherein the subject suffers from insomnia.
